(19) United States
(12) Patent Application Publication
(10) Pub. No.: US 2012/0197229 A1
(43) Pub. Date: Aug. 2, 2012

(54) SACRAL WOUND DRESSING AND METHOD OF MANUFACTURING A WOUND THERAPY DEVICE

(75) Inventors: John Buan, Maple Grove, MN (US); Alan Carlson, St. Paul, MN (US); Jason Buseman, Johnston, IA (US)

(73) Assignee: KALYPTO MEDICAL, INC., Mendota Heights, MN (US)

(21) Appl. No.: 13/197,518

(22) Filed: Aug. 3, 2011

Related U.S. Application Data
(60) Provisional application No. 61/370,637, filed on Aug. 4, 2010.

(51) Int. Cl. A61F 13/56; B23P 17/00; B23P 11/00

(52) U.S. Cl. 604/385.05; 29/428; 29/527.1

(57) ABSTRACT

A sacral wound therapy device which includes a backing material having an outer surface that is wipeable. A method of manufacturing a wound therapy device wherein a portion of a release liner remains on the backing material throughout at least a portion of the manufacturing process.

### RELATED APPLICATIONS

This application claims priority to U.S. Provisional Patent Application No. 61/370,637 filed on Aug. 4, 2010, the entirety of which is incorporated herein by reference.

### TECHNICAL FIELD

The present invention relates, in general, to a device and method for wound therapy that is capable of treating a variety of chronic and acute wound types, including, but not limited to, pressure ulcers, infection wounds, venous ulcers, arterial ulcers, diabetic ulcers, burn wounds, post amputation wounds, surgical wounds, and the like.

### BACKGROUND

Negative pressure therapy has been one method used for the treatment of a variety of different wounds by practitioners in the art. Conventional negative pressure therapy devices are generally large in size and often require the use of complicated equipment such as suction pumps, vacuum pumps and complex electronic controllers.

Furthermore, the wound dressings available with these existing systems can be cumbersome and time-consuming to apply. Once applied, the dressings require a great deal of care to deliver a successful treatment and do not tolerate much external abuse from the patient's activities of daily living or other factors. In the case of pressure ulcers of the sacrum, many of these patients need to be moved quite frequently to avoid the creation of new pressure sores and as an additional complicating factor, suffer both fecal and urinary incontinence. Both of these factors can stress the existing negative pressure dressing technology beyond its capability to function, resulting in treatment failure.

To remedy this, it is believed that in some instances, a negative pressure sacral wound dressing needs to be sufficiently wipeable, as well as have a moisture resistant seal to deliver appropriate therapy in the presence of urine and/or fecal matter.

In addition, while the present methods of manufacturing various wound therapy devices are presumably effective for their intended purposes, it is believed that a more economically beneficial method of manufacturing a negative pressure wound therapy device would be beneficial.

One such method to improve the manufacturing, as disclosed herein, is to leave a portion of a release liner on the backing material throughout the manufacturing process.

### BRIEF DESCRIPTION OF THE PRESENT INVENTION

In an embodiment of the present invention, a sacral wound therapy device includes a backing material having an outer surface and an inner surface, the outer surface being a wipeable material and the inner surface having an adhesive. A port hole is disposed in the backing material. The device may further include an absorptive pad disposed on the inner surface of the backing material such that a portion of the absorptive pad is disposed under the port hole. The absorptive pad is comprised of a material that passively draws exudates from a wound and is capable of retaining the exudates while the device is subjected to negative pressure.

In an embodiment of the present invention, the backing material is comprised of a first layer and a second layer. The first layer and second layer may be held together by an adhesive. The first layer may be a perforated EVA medical tape and the second layer may be a polyurethane medical tape. It is preferred that the backing material is semi-permeable.

In certain embodiments of the present invention, the device may also include a gasket disposed on the backing material distally between the absorptive pad and the edge. In some embodiments of the present invention, the gasket is disposed immediately adjacent the absorptive pad.

The gasket may be a hydrogel and having a width of approximately ¾ of an inch. In addition, the gasket may include a thickness of approximately 40 mils.

The absorptive pad has a shape and the gasket has a shape, and it is contemplated that the shape of the absorptive pad is substantially the same as the shape of the gasket. In a preferred embodiment the shape of the absorptive pad is a heart.

Further embodiments of the present invention include a wound interface layer disposed over a portion of the absorptive pad. The wound interface layer may be a silver plated mesh.

The device may include an adaptor with a fluid impermeable membrane disposed in the port hole.

A device such as described in one of the above embodiments is believed to be beneficial as it allows for fecal matter and other bodily fluids to be wiped of off the device. This would allow the negative pressure treatment to continue without changing the wound therapy device. This will allow a more constant treatment and also minimize the number of sacral wound therapy devices used on a patient.

Certain embodiments of the present invention are directed towards a method of manufacturing a negative pressure wound therapy device that generally includes providing a backing material with a shape having a perimeter and a release liner removably attached to a first side of the backing material by an adhesive, removing a first portion of the release liner such that a second portion of the release liner remains on the perimeter of the backing material, inserting an adaptor into the backing material, placing an absorptive pad onto the backing material such that a portion of the absorptive pad is disposed on top of the adaptor, placing a wound interface layer over the absorptive pad, and, disposing a gasket on the backing material between the perimeter of the backing material and the wound interface layer.

The step of disposing a gasket may be performed by pouring a gasket material and curing the gasket material.

In an embodiment of the invention, a second release liner may be attached to the backing material after the step of curing the gasket material, and, then the negative pressure wound therapy device may be cut into a desired shape. After cutting the device, the device may be sealed in a pouch.

A method such as described above is believed to be beneficial because it will allow more cost effective manufacturing of a negative pressure wound therapy device. Specifically, removing only a portion of the release liner provides structural support to the backing material as it is transported though the remaining steps of the manufacturing process. Without any portion of the release liner, the backing material lacks the structural integrity to be moved and operated on by hand and machine in the other steps.

Moreover, by pouring the gasket material, the manufacturing method provides a more economical method as it minimizes the waste of gasket material.

### BRIEF DESCRIPTION OF THE DRAWINGS

The present invention will become more fully apparent from the following description and appended claims, taken in conjunction with the accompanying drawings. Understanding that the accompanying drawings depict only typical embodiments, and are, therefore, not to be considered to be limiting of the scope of the present disclosure, the embodiments will be described and explained with specificity and detail in reference to the accompanying drawings as provided below.

FIG. 1 is a side partially exploded view of a sacral negative pressure wound therapy according to an embodiment of the present invention.

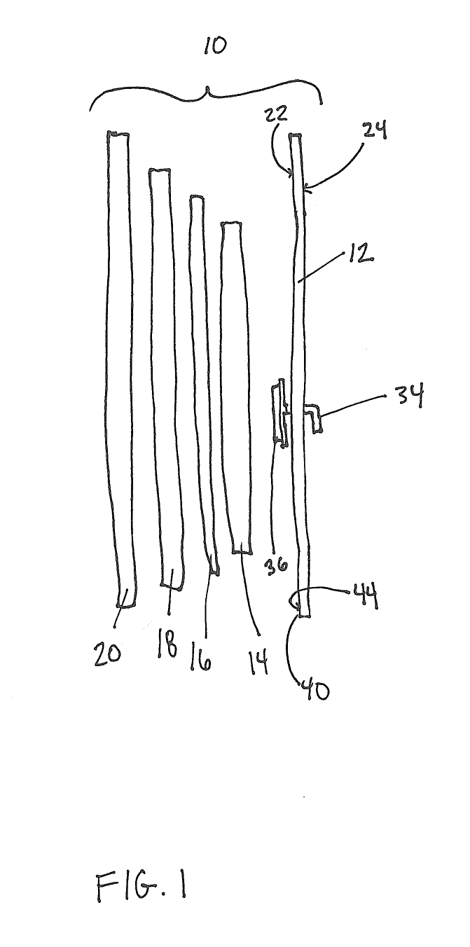

FIG. 2 is a side exploded view of a composite backing layer used in a sacral negative pressure wound therapy according to an embodiment of the present invention.

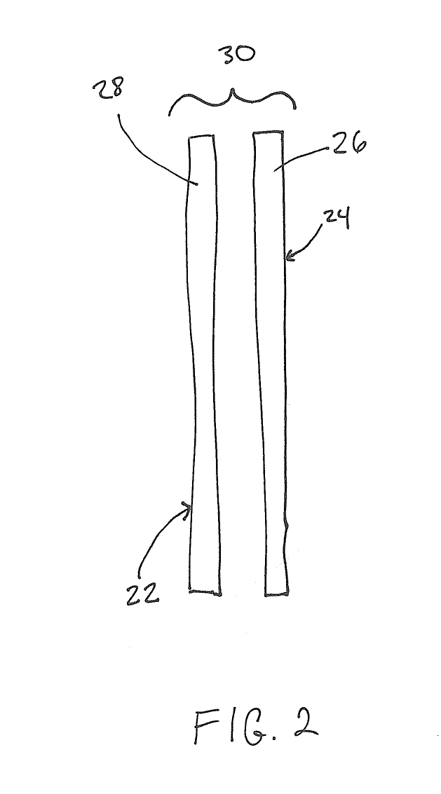

FIG. 3 is a top view of a sacral negative pressure wound therapy according to an embodiment of the present invention.

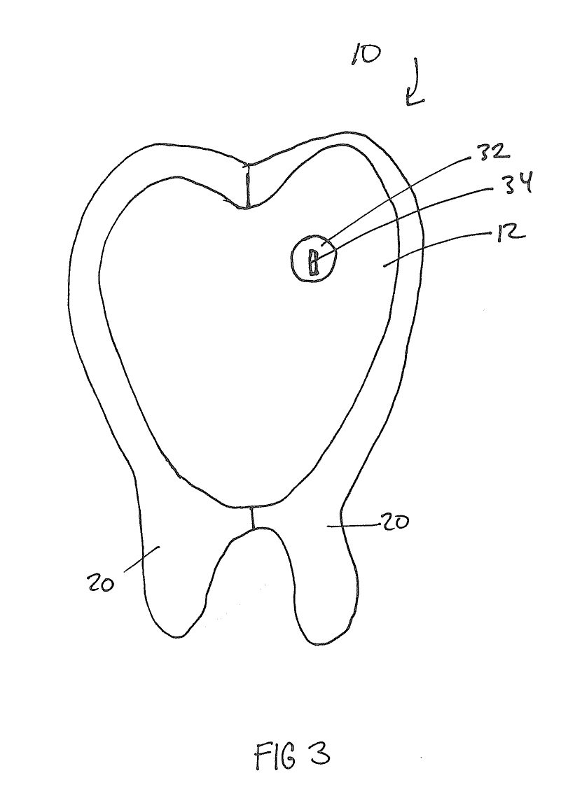

FIG. 4 is a bottom view of a sacral negative pressure wound therapy according to an embodiment of the present invention.

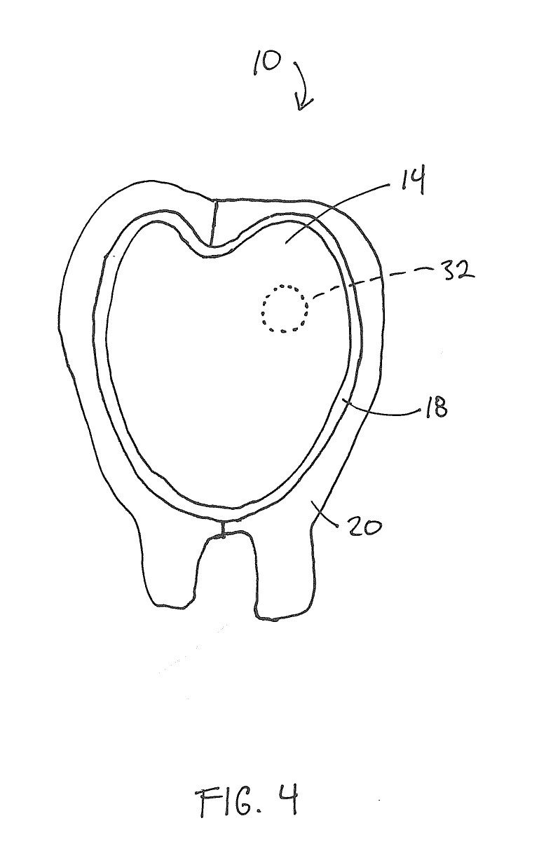

### DETAILED DESCRIPTION OF THE DRAWINGS

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

In the following description, numerous specific details are provided to provide a thorough understanding of embodiments of the invention. One skilled in the relevant art will recognize, however, that the invention can be practiced without one or more of the specific details, or with other methods, components, materials, and so forth. In other instances, well-known structures, materials, or operations such as vacuum sources are not shown or described in detail to avoid obscuring aspects of the invention.

Referring now to the attached Figures, a sacral wound therapy device 10 according to an embodiment of the present invention includes a backing material 12, an absorptive pad 14, a wound interface 16, a gasket 18, and a liner 20.

The backing material 12 has an inner surface 22 and an outer surface 24. The outer surface 24 is a wipeable material. The inner surface 22 includes an adhesive to hold the remaining components and to adhere the device 10 to the skin of a patient.

In a preferred embodiment, the backing material 12 is made from a first layer 26 and a second layer 28 held together by an adhesive to form a composite layer 30. It is preferred that the first layer 26 is a perforated EVA medical tape, such as Transpore™ #1527 available from 3M. Such a wipeable material will ensure that fecal matter and other bodily fluids are not absorbed through the backing material 12 requiring a changing of the wound therapy device 10 prior to its full use. Thus, by “wipeable” or “wipeable material” it is meant that the material will not absorb liquids or other material into and/or through the backing material 12.

It is also preferred in such an embodiment that the second layer 28 is a semi-permeable polyurethane medical film. The polyurethane film assures that the device 10 can function as a negative pressure device 10, i.e., the device 10 will hold a negative pressure for a reasonable period of time if air is removed from the device 10 when attached to the patient.

The wound therapy device 10 also includes a port hole 32 disposed in the backing material 12. The port hole 32 receives an adaptor 34 or valve, which allows negative pressure to be communicated from outside of the wound therapy device 10 to the wound. Typically, such an adaptor 34 is connected with tubing (not shown) to a negative pressure source like a pump (not shown). The adaptor 34 may also include a fluid impermeable membrane 36 to ensure that liquid/exudates removed from the wound do not migrate outside of the backing material 12.

The absorptive pad 14 is disposed on the inner surface 22 of the backing material 12 such that a portion of the absorptive pad 14 is disposed under the port hole 32. The absorptive pad 14 is comprised of a material that absorbs and retains the exudates while the device 10 is subjected to negative pressure. The absorptive pad 14 may also passively remove exudates from the wound when negative pressure is not being applied to the device 10.

The wound therapy device 10 may also include a gasket 18 disposed on the inner surface 22 of the backing material 12 distally between the absorptive pad 14 and the edge 40 of the backing material 12. Alternatively, the gasket 18 may be disposed immediately adjacent the absorptive pad 14.

Such gaskets 18 are described in more detail in pending U.S. patent application Ser. No. 12/455,013, the entirety of which is incorporated herein. The gasket 18 may be a hydrogel and have a width of approximately ¾ of an inch and have a thickness of approximately 40 mils.

The absorptive pad 14 may have a shape and the gasket 18 may have a shape, and the shape of the absorptive pad 14 may be substantially the same as the shape of the gasket. For example, the shape of the absorptive pad 14 may be a heart shape or an oval.

The device 10 further includes a wound interface layer 16 disposed on/over at least a portion of the absorptive pad 14 to prevent tissue from growing on and adhering to the absorptive pad 14. The wound interface layer 16 may be a silver plated mesh.

It is preferred that a skirt 44, or a section of the inner surface 22 of the backing material 14 between the edge 40 of same and the gasket 38, is provided to allow the wound therapy device 10 to be placed and secured onto the skin of a patient. Prior to placement, the wound therapy device 10 preferably includes a liner 46 over at least the skirt 44. In a preferred embodiment, the liner 46 is a two piece liner to allow for easy placement and positioning on the patient's skin.

The present invention also provides a method of manufacturing of a negative pressure wound therapy device. According to this aspect of the invention, the method includes the steps of: providing a backing material with a shape having a perimeter and a release liner removably secured to a first side of the backing material by an adhesive; removing a first portion of the release liner such that a second portion of the release liner remains on the perimeter of the backing material; inserting an adaptor into the backing material; placing an absorptive pad onto the backing material such that a portion of the absorptive pad is disposed on top of the adaptor; placing a wound interface layer over the absorptive pad; and, disposing a gasket on the backing material between the perimeter of the backing material and the wound interface layer.

Other backing materials other than the two material configuration described above with respect to the sacral wound therapy device may be used.

The method may also include the steps of pouring a gasket material, and curing the gasket material to form the gasket.

The method may also include the steps of applying a second release liner to the backing material after the step of disposing a gasket, and cutting the negative pressure wound therapy device into a desired shape.

The method of may also include the step of sealing the negative pressure wound therapy device in a pouch after the step of cutting the backing material.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure provided herein. In other words, various modifications and improvements of the embodiments specifically disclosed in the description above are within the scope of the appended claims. Note that elements recited in means-plus-function format are intended to be construed in accordance with 35 U.S.C. §112 ¶6. The scope of the invention is therefore defined by the following claims.

1. A sacral wound therapy device comprising:
a backing material having an outer surface and an inner surface, the outer surface being a wipeable material and the inner surface having an adhesive;
a port hole disposed in the backing material; and,
an absorptive pad disposed on the inner surface of the backing material such that a portion of the absorptive pad is disposed under the port hole, the absorptive pad comprised of a material that passively draws exudates from a wound and retains the exudates while the device is subjected to negative pressure.

2. The device of claim 1 wherein the backing material is comprised of a first layer and a second layer.

3. The device of claim 2 wherein the first layer and second layer are held together by an adhesive.

4. The device of claim 1 further comprising a gasket disposed on the inner side of the backing material between the absorptive pad and an edge of the backing material.

5. The device of claim 4 further comprising the absorptive pad having a shape and the gasket having a shape, and wherein the shape of the absorptive pad is substantially the same as the shape of the gasket.

6. The device of claim 5 further comprising the shape of the absorptive pad being a heart shape.

7. The device of claim 4 further comprising the gasket being a hydrogel and having a width of approximately ¾ of an inch.

8. The device of claim 7 further comprising the gasket having a thickness of approximately 40 mils.

9. The device of claim 4 further comprising the gasket being disposed immediately adjacent the absorptive pad.

10. The device of claim 3 further comprising a wound interface layer disposed over at least a portion of the absorptive pad.

11. The device of claim 10 wherein the wound interface layer is a silver plated mesh.

12. The device of claim 1 wherein the backing material is semi-permeable.

13. The device of claim 1 wherein the port hole further comprises an adaptor with a fluid impermeable membrane.

14. The device of claim 2 wherein the first layer is a perforated EVA medical tape and the second layer is a polyurethane medical tape.

15. The device of claim 4 wherein the backing material has a skirt and a two piece release liner is disposed on the skirt.

16. A method of manufacturing a negative pressure wound therapy device comprising the steps of:
providing a backing material with a shape having a perimeter and a release liner removably secured on a first side of the backing material by an adhesive;
removing a first portion of the release liner such that a second portion of the release liner remains on the perimeter of the backing material;
inserting an adaptor into the backing material;
placing an absorptive pad onto the backing material such that a portion of the absorptive pad is disposed on top of the adaptor;
placing a wound interface layer over the absorptive pad; and,
disposing a gasket on the backing material between the perimeter of the backing material and the wound interface layer.

17. The method of claim 16 wherein the step of disposing a gasket comprises the steps of:
pouring a gasket material; and,
curing the gasket material.

18. The method of claim 16 further comprising the steps of:
applying a second release liner to the backing material after the step of curing the gasket material; and,
cutting the negative pressure wound therapy device into a desired shape.

19. The method of claim 18 further comprising the steps of:
sealing the negative pressure wound therapy device in a pouch after the step of cutting the negative pressure wound therapy device.

20. The method of claim 18 wherein the second release liner is a two piece release liner.
